(19) United States
(12) Patent Application Publication
(10) Pub. No.: US 2009/0182228 A1
(43) Pub. Date: Jul. 16, 2009

(54) WIRELESS ULTRASOUND SYSTEM DISPLAY

(75) Inventor: Michael Pierce, Carnation, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/575,372

(22) PCT Filed: Sep. 13, 2005
(86) PCT No.: PCT/IB05/52994

§ 371 Date: Mar. 15, 2007

Related U.S. Application Data
(60) Provisional application No. 60/610,939, filed on Sep. 17, 2004.

(51) Int. Cl. A61B 8/00

(52) U.S. Cl. 600/437

(57) ABSTRACT

Ultrasound imaging arrangement including an ultrasound scanner, an ultrasound imaging system arranged to control the scanner and at least one display physically separated from the imaging system. A system is provided to enable the transmission of images from the imaging system to each display using IEEE standard 802.15.3. For example, the imaging system and each display may include a network interface card/video card capable of utilizing IEEE standard 802.15.3. The absence of a physical connection between the display(s) and the imaging system allows the display(s) to be freely positionable as desired by the person conducting the examination without limitations imposed by the placement of the imaging system. Multiple displays can be provided, all receiving and displaying the same images.

The present invention relates generally to medical diagnostic imaging arrangements, such as ultrasound imaging arrangements, and more particularly to ultrasound imaging arrangements including wireless displays.

An ultrasound imaging arrangement generally includes an ultrasound scanner which is placed on a patient being scanned, a control unit coupled to the ultrasound scanner which controls ultrasound signals generated and received by the scanner and converts the received signals into images and a main display on which the ultrasound images are presented. The main display is physically connected to the control unit, e.g., by a cable, and is typically positioned on or alongside the control unit.

One problem with the conventional ultrasound arrangement relates to the placement of the control unit and main display. Since the sonographer is situated alongside the patient and must have the control unit (which has main display connected thereto) within reach, the control unit is also typically placed alongside the patient on the same as the sonographer. As such, the sonographer must repeatedly shift his view, i.e., turn his head, from the ultrasound scanner placed on the patient to the main display in order to alternately adjust the position the scanner and view the images being formed. This results in user fatigue. Moreover, since the display is placed in a position in which the sonographer can easily view the images thereon, it is difficult if not impossible for the patient to simultaneously view the images.

It would be desirable to enable the main display to be separate from the control unit in order to avoid imposing limitations on the placement of the main display.

U.S. Pat. No. 6,440,072 describes an ultrasound imaging arrangement in which ultrasound examination data is transferred wirelessly from an imaging system to a portable computing device, such as a personal digital assistant (PDA).

It is an object of the present invention to provide a new and improved ultrasound imaging arrangement in which a display can be placed in any position relative to the patient, user and control unit. As such, the display is not limited to a position on or alongside the control unit and can be placed in an optimally ergonomic position.

It is another object of the present invention to provide a new and improved system and method for wirelessly transferring ultrasound examination images from an ultrasound imaging arrangement to one or more physically separated displays.

In order to achieve these objects and others, an ultrasound imaging arrangement in accordance with the invention includes an ultrasound scanner, an ultrasound imaging system arranged to control the scanner and at least one display physically separated from the imaging system. A system is provided to enable the transmission of images from the imaging system to each display using IEEE standard 802.15.3. For example, the imaging system and each display may include a network interface card/video card capable of applying IEEE standard 802.15.3. The absence of a physical connection between the display(s) and the imaging system allows the displays to be freely positionable as desired by the person conducting the examination without limitations imposed by the placement of the imaging system.

Thus, ultrasound examinations can be conducted with the imaging system on one side of a patient and the display on an opposite side to enable the sonographer on the side with the imaging system to view both the patient and the display in a common line of sight. The sonographer does not need to turn his head when adjusting the position of the scanner and viewing the displayed images. This significantly improves the sonographer's ability to conduct the examination while reducing fatigue.

In addition, other displays can be placed within the range of the network interface card/video card of the imaging system, including one which is in front of and viewable by the patient. The patient and sonographer can thus both easily view the ultrasound images, each on a separate, dedicated display. An additional display can be placed in another room, e.g., in a doctor's office for viewing by the doctor or family members.

### BRIEF DESCRIPTION OF THE DRAWINGS

The invention, together with further objects and advantages thereof, may best be understood by reference to the following description taken in conjunction with the accompanying drawings wherein like reference numerals identify like elements.

FIG. 1 is a schematic diagram of an ultrasound imaging arrangement m in accordance with the invention.

FIG. 2 is a schematic showing the use of multiple wireless displays in accordance with the invention.

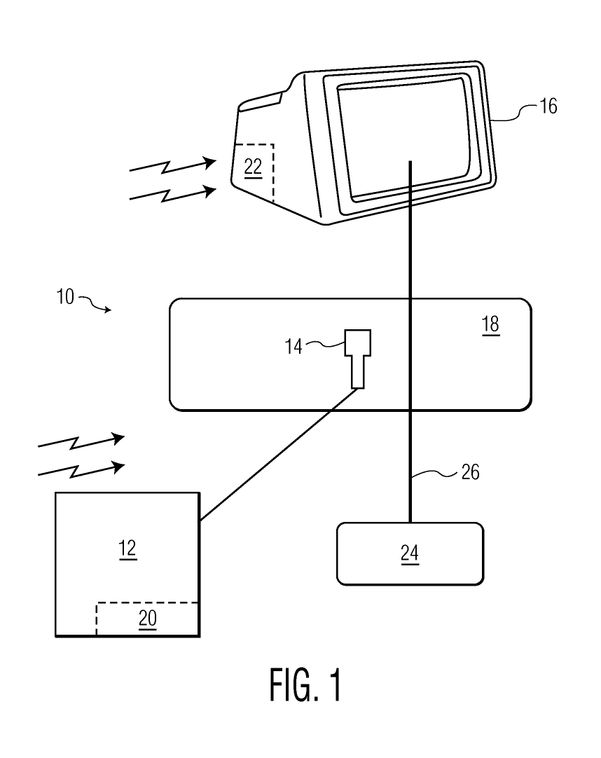

Referring to FIG. 1, an ultrasound imaging arrangement 10 in accordance with the invention includes an ultrasound imaging system 12, an ultrasound scanner 14 coupled to the imaging system 12 and a wireless display 16 physically separated from the imaging system 12. Imaging system 12 includes conventional components such as a processor, electrical and mechanical subsystems for controlling operation and use of the scanner 14 and user interface components such physical keys in the form of a keyboard, buttons, slider potentiometers, knobs, switches and a trackball. The scanner 14 is typically placed on a patient 18 during an examination.

In accordance with the invention, the ultrasound imaging arrangement 10 also includes a network interface card/video card 20 arranged in the imaging system 12 and which receives images generated by the processor in the imaging system 12 and converts the images to a wireless stream of data representative of the images and transmits this stream of data. The wireless stream of data is received by a network interface card/video card 22 arranged in the display 16 which converts the stream of data into images and displays the images.

The wireless coupling between the imaging system 12 and the display 16 allows for virtually unlimited placement of the display 16 relative to the imaging system 12, the only limitation being the range of the transmission of image data from the network interface card/video card 20 in the imaging system 12. Thus, as shown in FIG. 1, the display can be arranged on an opposite side of the patient 18 from the sonographer 24. The line of sight of the sonographer (represented by the line designated 26) thus includes both the patient 18 and the display 16 so that the sonographer does not have to shift his view from the ultrasound scanner on the patient to the display in order to alternately position the scanner and view the images being formed as in conventional ultrasound arrangements. Rather, in the invention, the sonographer 24 can continually look in the direction of the display 16 while at the same time viewing the position of the scanner 14 and adjustments thereto.

Power can be supplied to the display 16 through an electrical connection leading to an outlet separate from a main cart on which the imaging system 12 is arranged.

The wireless transmission of data representing constantly changing images is often problematic since the images must be transmitted at a high bandwidth. This problem is overcome in the invention by using the IEEE 802.15.3 standard which provides for data transmission at rates of 200 and 400 Mbits/s at ranges of 10 meters or less (about 30 ft). This standard is part of the so-called WPAN Personal Area Networks and is a UWB ultra wide-band communication standard that has a relatively high data transfer capacity at frequencies which do not interfere with current technology. More specifically, the band width range includes 53.3, 55, 80, 106.7, 110, 160, 200, 320 and 480 Mbits/s while the operating frequency is in a range from about 3.1 GHz to about 10.6 GHz (UWB), with 122 sub-bands.

In one embodiment of the invention, IEEE 802.15.3 standard will apply the H.264/AVC compression standard which has been used to transfer HDTV quality signals over the Internet. Additional details about the H.264/AVC compression standard and the construction of a network interface card/video card capable of applying the standard to compress images are found in “The Emerging H.264/AVC Standard” by R. Schafer et al., EBU Technical Review, January 2003, incorporated by reference herein.

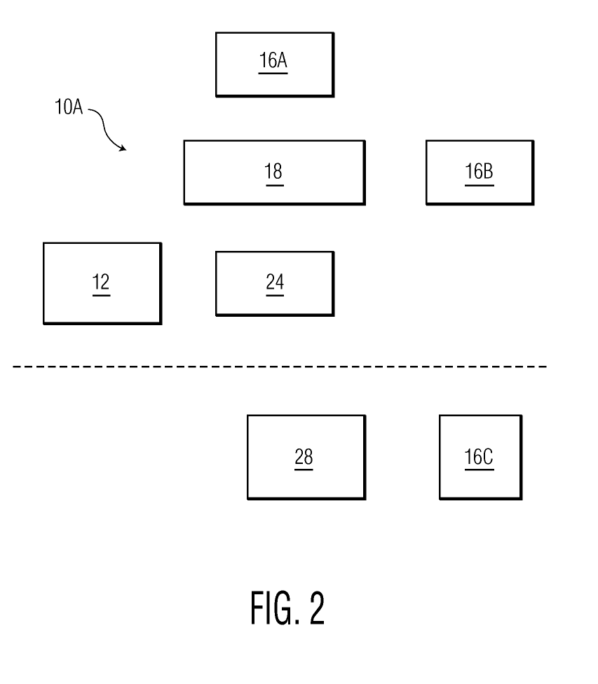

An advantage resulting from the use of the 802.15.3 standard is that multiple wireless displays 16A, 16B, 16C can be used in an ultrasound arrangement 10A in accordance with the invention as shown in FIG. 2. Each display 16A, 16B, 16C would receive the same stream of data from the network interface card/video card 20 in the imaging system 12 and therefore display the same images. With multiple displays 16A, 16B, 16C, it is possible to arrange one display 16A to be viewed by the sonographer, another display 16B by the patient and another display 16C by another individual 28, such as a doctor or the patient's family member, who might be outside of the examination room (but within the 10 meter transmission range of the network interface card/video card 20).

Although the wireless displays and method for wireless transmission of images thereto in accordance with the invention is described for use in an ultrasound imaging system, the same displays and transmission method can also be used in other types of medical diagnostic imaging systems, such as an MRI system, an X-ray system, an electron microscope, a heart monitor system, and the like. The displays and wireless transmission method can also be used in a home setting for transmitting images to one or more televisions or computer monitors.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. An ultrasound imaging arrangement (10), including:
an ultrasound scanner (14);
an ultrasound imaging system (12) arranged to control said scanner (14); and
at least one display (16, 16A, 16B, 16C) physically separated from said imaging system (12), said imaging system (12) and each of said at least one display (16, 16A, 16B, 16C) including means (20, 22) for enabling transmission of images from said imaging system (12) to each of said at least one display (16, 16A, 16B, 16C) using IEEE standard 802.15.3.

2. The arrangement of claim 1, wherein said means (20, 22) comprise a network interface card/video card (20) arranged in connection with said imaging system (12) and a network interface card/video card (22) arranged in connection with each of said at least one display (16, 16A, 16B, 16C).

3. The arrangement of claim 1, wherein said at least one display (16, 16A, 16B, 16C) comprises a plurality of displays (16A, 16B, 16C) each freely positionable relative to the other of said displays (16A, 16B, 16C).

4. A method for conducting ultrasound examinations, comprising:
positioning an ultrasound scanner (14) coupled to an ultrasound imaging system (12) on the patient (18);
positioning a first display (16, 16A) in a position viewable by the person conducting the examination; and
transmitting images from the imaging system (12) to the first display (16, 16A) using IEEE standard 802.15.3.

5. The method of claim 4, wherein the transmitting step comprises arranging a first network interface card/video card (20) in connection with the imaging system (12) and a second network interface card/video card (22) in connection with the first display (16, 16A).

6. The method of claim 4, wherein the first display (16, 16A) is positioned in front of the person (24) conducting the examination in a common line of sight (26) with the patient (18) being examined.

7. The method of claim 6, further comprising:
positioning a second display (16B) in a position in front of the patient to be easily viewable by the patient (18); and
transmitting images from the imaging system (12) to the second display (16B) using IEEE standard 802.15.3.

8. The method of claim 7, further comprising:
positioning a third display (16C) in a different room than the patient (18); and
transmitting images from the imaging system (12) to the third display (16C) using IEEE standard 802.15.3.

9. A method for conducting ultrasound examinations, comprising:
arranging an ultrasound imaging system (12) on a first side of a patient (18);
positioning an ultrasound scanner (14) coupled to the imaging system (12) on the patient (18);
positioning a first display (16, 16A) on a second side of the patient (18) opposite the first side to enable a person (24) to conduct the examination from the first side while viewing the patient (18) and the first display (16, 16A) in a common line of sight (26) toward the second side; and
transmitting images from the imaging system (12) to the first display (16, 16A) using IEEE standard 802.15.3.

10. The method of claim 9, wherein the transmitting step comprises arranging a first network interface card/video card (20) in connection with the imaging system (12) and a second network interface card/video card (22) in connection with the first display (16, 16A).

11. The method of claim 9, further comprising:
positioning a second display (16B) in a position in front of the patient (18) to be easily viewable by the patient (18); and
transmitting images from the imaging system (12) to the second display (16B) using IEEE standard 802.15.3.

12. The method of claim 11, further comprising:
positioning a third display (16C) in a different room than the patient (18); and
transmitting images from the imaging system (12) to the third display (16C) using IEEE standard 802.15.3.
